(19) United States
(12) Patent Application Publication
(10) Pub. No.: US 2020/0368028 A1
(43) Pub. Date: Nov. 26, 2020

(54) MEDICAL IMPLANTS COMPRISING ANTI-INFECTIVE SURFACES

(71) Applicant: DEPUY IRELAND UNLIMITED COMPANY, County Cork (IE)

(72) Inventors: VICTOR Victor BELLIDO-GONZALEZ, Yorkshire (GB); Niki GOSLING, Yorkshire (GB); Edward LATTER, Yorkshire (GB); Dermot MONAGHAN, Yorkshire (GB); Rasmita RAVAL, St Anthony's Road (GB); Jonathan THOMPSON, Yorkshire (GB)

(21) Appl. No.: 16/966,288

(22) PCT Filed: Feb. 4, 2019
(86) PCT No.: PCT/EP2019/052659

§ 371 Date: Jul. 30, 2020

(30) Foreign Application Priority Data
Feb. 9, 2018 (GB) 1802109.7

(51) Int. Cl. A61F 2/30

(52) U.S. Cl. CPC A61F 2/30767; A61F 2/3094; A61F 2310/00389; A61F 2310/00023

(57) ABSTRACT

A medical implant comprising: ⋅an implant body configured for use as a medical implant, ⋅a surface on the implant body, ⋅wherein the surface comprises a plurality of projections (40), each projection having a base proximal to the implant body, a peak distal to the implant body, and a side wall extending from the base to the peak, ⋅wherein the surface has a peak density in a range 50 to 500 peaks per 11 m², and ⋅wherein the projections are tapered such that a width at the peak of each projection is less than a width at the base of each projection.

### FIELD OF INVENTION

The present invention relates to medical implants comprising anti-infective surfaces. Particular embodiments relate to medical implants such as prosthetic joints including hip joints, knee joints, shoulder joints, and the like and components thereof.

### BACKGROUND OF INVENTION

One problem with medical implants is biofilm formation on their surface leading to infection. Bacterial cells can attach to a surface of the medical implant. The bacterial cells can proliferate and produce extracellular polysaccharide slime (EPS) forming a matrix in which the bacterial cells are disposed. This can continue until the matrix erupts releasing planktonic cells resulting in infection. The best defence against such biofilm formation and resultant infection is for host tissue coverage of the medical implant.

In light of the above, it is desirable to provide a medical implant having a surface which has reduced bacterial adhesion while promoting, or at least not adversely effecting, host cell adhesion. Whilst a substantial amount of research has been carried out investigating the impact of the chemical nature of the surface, a recent trend has been to elucidate the role that surface topography plays in the cell attachment process. The successful design of an implant should take into consideration the positive attributes directed towards host cell (osteoblast) adhesion and likewise, limit the deleterious effect of bacterial colonization. In the prior art these dual targets have been considered separately which largely reflects the available literature and focus of these studies. The adhesion of osteoblasts and bacteria has also been compared on the same surface but these have not been done in direct competition and so it is unclear how the presence of one type of cell may impact on the adhesion of the other.

One such prior art document is by Izquierdo-Barba et al., Acta Biomaterialia, 15, 2015, 20-28. Izquierdo-Barba et al. have disclosed nano-columnar coatings with selective behaviour towards osteoblast and Staphylococcus aureus proliferation. Such coatings comprise a high density of nano-columnar structures which impair bacterial adhesion. Osteoblast adhesion to such surfaces is also reduced but not to the same extent.

One problem which the present inventors have considered is how to provide a nano-structured surface which impairs bacterial adhesion (small rigid cells) without also reducing osteoblast adhesion (relatively large, deformable cells).

Another problem which the present inventors have considered is the mechanical robustness of such nano-columnar surfaces. Nano-columnar structures are weak and can be prone to fragmentation in use resulting in nanoparticles being released into a patient's system. Such nanoparticles have potential to cause adverse effects on organs, tissue, and cells. In addition, physical damage to the topography of the surface during storage of the implant or in use will adversely affect the functional performance of the surface in terms of reducing bacterial adhesion.

### SUMMARY OF INVENTION

A medical implant is described herein which comprises:
an implant body configured for use as a medical implant,
a surface on the implant body,
wherein the surface comprises a plurality of projections, each projection having a base proximal to the implant body, a peak distal to the implant body, and a side wall extending from the base to the peak,
wherein the surface has a peak density in a range 50 to 500 peaks per μm², and
wherein the projections are tapered such that a width at the peak of each projection is less than a width at the base of each projection.

The present inventors have moved away from prior art nano-columnar surfaces to provide surfaces which have a reasonably high peak density but which also have tapered projections. The high peak density still provides a surface which has reduced adhesion for small, rigid bacterial cells. However, such a surface also provides an increased surface area accessible to larger, deformable host cells and thus adhesion of such host cells is not reduced to the same extent as for a nano-columnar surface structure.

Furthermore, tapered projections are more mechanically robust than columnar structures thus reducing the potential for the projections to fragment in use. As such, the possibility of nanoparticles causing adverse local or systemic tissue reactions is reduced. Further still, an increase in mechanical robustness provides an implant surface topology which is less likely to be damaged leading to a reduced functional performance of the surface in terms of resisting bacterial adhesion.

Within the peak density range of 50 to 500 peaks per μm², the peak density may be greater than 100, 150, or 200 peaks per μm², less than 400, 300, or 250 peaks per μm², or a range defined by any combination of these lower and upper limits. For example, one such surface which has been found to be particularly effective at resisting proliferation of bacterial cells during dual incubation of bacteria and host cells has a peak density between 200 and 250 peaks per μm². If the peak density is too low then bacterial cell adhesion can increase. If the peak density is too high then the individual projections become too narrow and fragile. Furthermore, the surface area accessible to larger, deformable host cells can reduce thus reducing adhesion of host cells.

The tapering of the projections can be defined such that the width at the base of each projection is at least 1.2, 1.4, 1.6, 1.8 or 2 times the width of each projection at ⅘^{th} of a height of each projection. This is distinct from columnar projections which have an approximately constant diameter from base to tip. Typical prior art column diameters lie in a range 30 to 100 nm. The tapering of the present invention allows for the provision of a smaller tip and/or a larger base which falls outside this range to combine better mechanical robustness with better adhesion characteristics.

Furthermore, the peak of each projection can be rounded, e.g. by etching. For example, the rounded peak of each projection may have a radius of curvature in a range 5 nm to 200 nm, optionally 15 to 100 nm. On the face of it, rounded peaks may be expected to increase adhesion of bacteria via increased surface area. However, the combination of a relatively high peak density in combination with rounded peaks, and the fact that bacterial bodies are relatively rigid, and can be spherical or rod-like, actually results in low bacterial adhesion. Furthermore, the surface is more amenable to adhesion of larger, deformable host cells which can result in an overall improvement in performance.

The projections may have a height from base to peak in a range 30 nm to 90 nm. Within the height range of 30 nm to 90 nm, the height of the projections may be greater than 40 nm, less than 80 nm, 70 nm, 60 nm, 50 nm, or 45 nm, or a range defined by any combination of these lower and upper limits. Prior art columnar structures typically have a height between 100 nm and 300 nm. In contrast, lower height, tapered projections as described herein are more mechanically robust and can allow better adhesion of host cells while still resisting bacterial adhesion. Further still, etching of the surface can simultaneously reduce peak height and also provided rounded peaks so as to provide an advantageous combination of features for promoting host cell adhesion while resisting bacterial adhesion.

The surface may have a surface roughness (Ra) in a range >5 nm to 18 nm. Within this surface roughness range, the surface roughness (Ra) may be greater than 6 nm, 7 nm, or 7.5 nm, less than 14 nm, 12 nm, 10 nm, or 9 nm, or within a range defined by any combination of these lower and upper limits. This contrasts with prior art columnar surfaces which typically have a surface roughness less than 5 nm. In this regard, it may be noted that surface parameter features are interrelated and thus a change in the shape of the projections (columnar to tapered) can also result in a change to the optimum surface roughness required to promote adhesion of large, deformable host cells while resisting adhesion of small, rigid bacterial cells.

Yet another difference between columnar prior art surfaces and the tapered projections of surfaces as described herein is the orientation of the projections. Columnar structures are generally formed by a glancing angle deposition technique which results in columnar projections with an inclination angle up to 30°. In contrast, the tapered structures of the present invention can be formed by coating and etching techniques which result in projections extending vertically from the surface of the implant body. This can lead to a more symmetric surface structure which is more readily and reproducibly fabricated, particularly on three dimensional implant body structures with non-planar surfaces.

When considering the adhesion of bacteria and osteoblasts, it has been found that surface order can also be important and thus the importance of including spatial parameters such as skewness and kurtosis becomes apparent. For example, surfaces can have similar average roughness but are significantly different with respect to their spatial parameters. Skewness is a measure of asymmetry in a histogram of projection height distribution whereas kurtosis is a measure of whether the surface is peaked or flat relative to the mean. Both are well defined mathematical parameters. Furthermore, both can vary significantly depending on the specific tapered projection structure of surfaces as described herein. For example, surfaces may have a kurtosis in a range 2.50 to 4.00. Within this range the kurtosis of the surface may be greater than 2.6, 2.7, 2.8, or 2.9, less than 3.8, 3.6, 3.4, or 3.2, or within a range defined by any combination of these lower and upper limits. Additionally, surfaces may have a skewness in a range −0.20 to +0.30. Within this range, the skewness of the surface may be greater than −0.10, −0.05, 0.00, or 0.05, less than 0.25, 0.20, 0.15, or 0.10, or within a range defined by any combination of these lower and upper limits. These values of kurtosis and skewness relate to the form of the tapered projections and thus relate to both the mechanical robustness and the adhesive properties of the surface for bacteria and host cells.

Surfaces as described herein can be formed by a coating on an implant body. However, it is also envisaged that such surfaces can be formed directly into the implant body by, for example, etching. The surfaces can be formed of titanium or a titanium alloy such as a titanium aluminium vanadium alloy. Such materials are consistent with those used presently for implant bodies such as prosthetic joints and components thereof.

### BRIEF DESCRIPTION OF THE DRAWINGS

Embodiments of the present invention are described by way of example only with reference to the accompanying drawings in which:

FIG. 1(a) shows a schematic illustration of how a small, rigid bacteria cell and a large, deformable host (mammalian) cell interact with a prior art nano-columnar surface;

FIG. 1(b) shows a sample of a prior art nano-columnar coating;

FIG. 2(a) shows a schematic illustration of how a small, rigid bacteria cell and a large, deformable host (mammalian) cell interact with a tapered projection surface as described herein;

FIG. 2(b) shows a sample of a coating as described herein;

FIGS. 3 to 6 show four examples of surfaces as described herein (referred to as runs 18 to 21 respectively);

FIG. 7 shows the percentage surface coverage (hMSCs) following a race to the surface co-culture test with S. aureus for surfaces shown in FIGS. 3 to 6;

FIG. 8 shows the density of peaks for the four surfaces shown in FIGS. 3 to 6;

FIG. 9 shows the average feature size (nm) for the four surfaces shown in FIGS. 3 to 6;

FIG. 10 shows the surface roughness Ra (nm) for the four surfaces shown in FIGS. 3 to 6;

FIGS. 11(a) and 11(b) illustrated the surface parameters of skewness and kurtosis respectively;

FIG. 12 shows kurtosis data for the four surfaces shown in FIGS. 3 to 6;

FIG. 13 shows skewness data for the four surfaces shown in FIGS. 3 to 6;

FIG. 14 illustrates a sputtering coating technique;

FIG. 15 illustrates surface processing steps including etching and coating; and

FIG. 16 shows a stem of a prosthetic hip joint to which surface processing as described herein has been applied.

### DETAILED DESCRIPTION

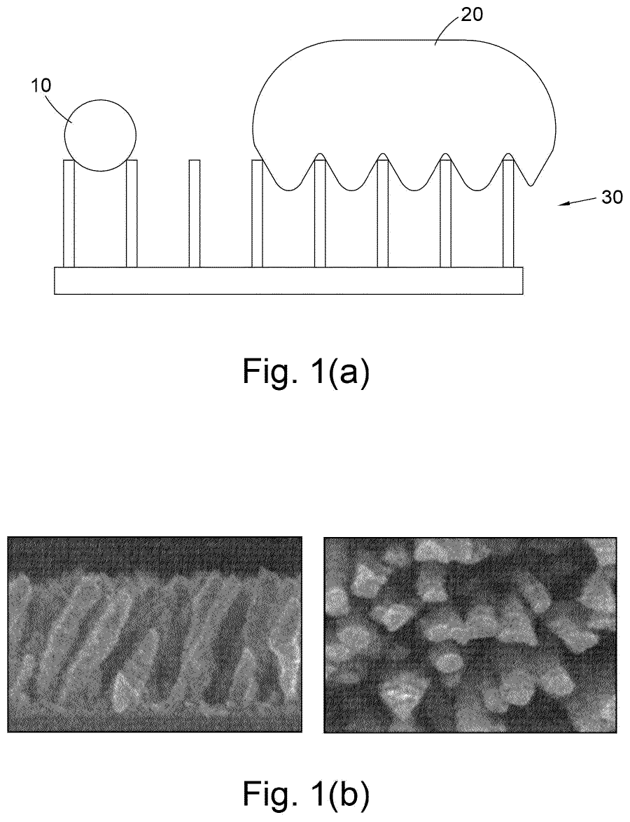

FIG. 1(a) is a schematic illustration of how a small, rigid bacteria cell 10 and a large, deformable host (mammalian) cell 20 interact with a prior art nano-columnar surface 30. The nano-columnar surface comprising a plurality of column-like projections 30. Typical prior art nano-columnar coatings have a column height between 100 nm and 300 nm, a column diameter in a range 30 to 100 nm, and a surface roughness Rₐ of less than 5 nm. The columnar structures are generally formed by a glancing angle deposition technique which results in columnar projections with an inclination angle up to 30° relative to vertical. The inclination angle is not shown in the schematic of FIG. 1 but can be seen in the image of an actual prior art nano-columnar coating shown in FIG. 1(b).

As can be seen in FIG. 1(a), a bacterial cell is small with a rigid cellular wall and has a low contact surface area with such a nano-columnar coating. As such, adhesion of bacterial cells is low. In contrast, host cells such as human Mesenchymal stem cells (hMSCs) are large and deformable and extend partially down the side walls of the columnar projections thus having a larger contact surface area and a higher associated adhesion. That said, the adhesion of host cells to such a nano-columnar surface is still reduced when compared to a planar surface. Furthermore, as described in the background and summary of invention sections, the thin columnar projections are relatively fragile and can be damaged and fragmented during storage or in use. This may result in nanoparticles being released into a patient's system and may cause adverse effects on organs, tissue, and cells. In addition, physical damage to the topography of the surface during storage of the implant or in use can adversely affect the functional performance of the surface in terms of reducing bacterial adhesion.

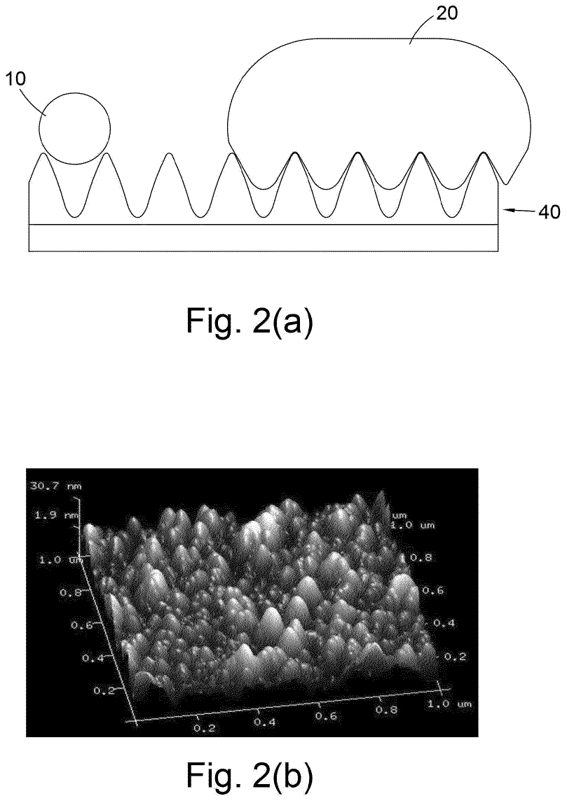

FIG. 2(a) shows a schematic illustration of how a small, rigid bacteria cell 10 and a large, deformable host (mammalian) cell 20 interact with a tapered projection surface 40 as described herein. The surface has a peak density in a range 50 to 500 peaks per μm² and the projections are tapered such that a width at the peak of each projection is less than a width at the base of each projection. An actual sample comprising such a surface structure is shown in FIG. 2(b).

As with the prior art nano-columnar structure, a bacterial cell is small and rigid and has a low contact surface area with such a surface resulting in low bacterial cell adhesion. Host cells such as human Mesenchymal stem cells (hMSCs) are large and deformable and extend partially down the side walls of the columnar projections thus having a larger contact surface area and a higher associated adhesion. However, in contrast to the nano-columnar structure of FIG. 1, the tapered projections increase the contact surface area to a level not far removed from a planar surface such that adhesion of the host cell is not significantly reduced. As such, the ratio of host cell adhesion to bacterial cell adhesion is improved when compared with the nano-columnar structure. Further still, the smaller tapered projections are more mechanically robust and less prone to damage and fragmentation. This improves the reliability of functional performance in use and reduces the risk of fragmented nanoparticles causing adverse local or systemic tissue reactions.

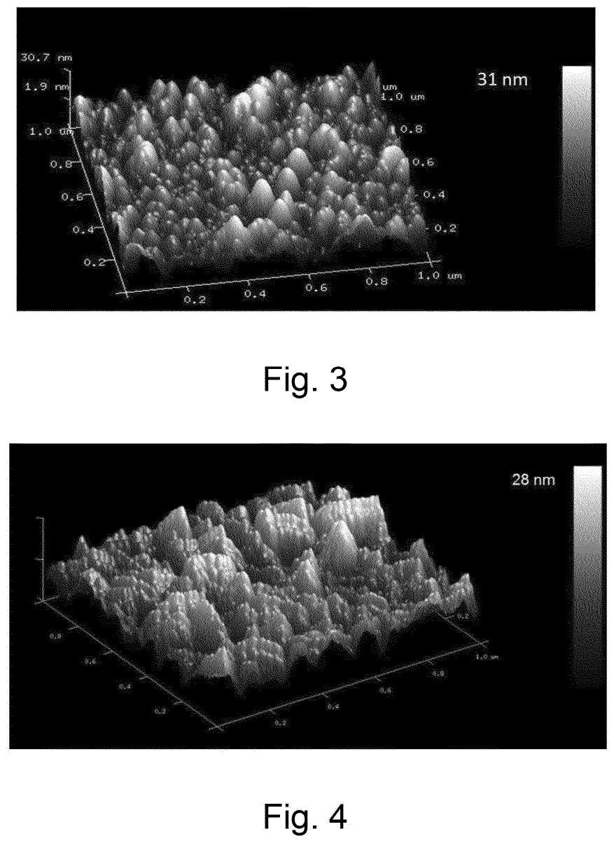

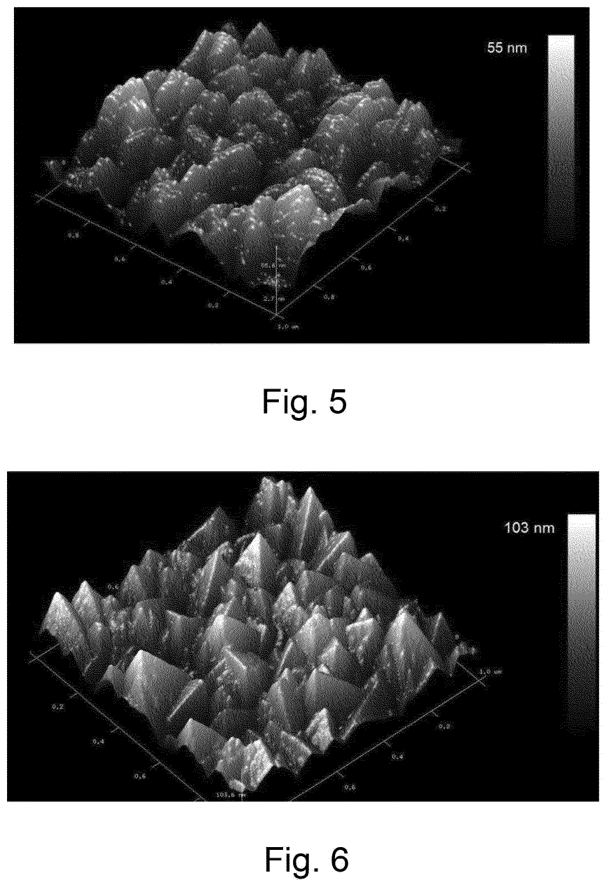

FIGS. 3 to 6 show four examples of surfaces as described herein (referred to as runs 18 to 21 respectively). All the surfaces share the common feature of having a peak density in a range 50 to 500 peaks per μm² and the projections are tapered such that a width at the peak of each projection is less than a width at the base of each projection. The surfaces vary in terms of their more detailed structure as further described later in this specification.

A test was developed to analyze the adhesion of bacteria on the prepared surfaces using a modified Atomic Force Microscopy (AFM) probe. The technique requires adhesion of a single bacterium on to the AFM probe, the probe is then brought into contact with the surface coating allowing the bacteria to form an attachment to the surface and then the probe is removed. The force required to remove the bacteria was measured and recorded. A study was conducted investigating the adhesion of S. epidermidis and P. aeruginosa to titanium alloy (Ti6Al4V) surfaces following either a polished surface finish or magnetron sputter coating process (Runs 18 and 21). Results are shown in Table 1 below.

| S. epidermidis | | | P. aeruginosa | | |
|---|---|---|---|---|---|
| | Proportion | | | Proportion | |
| | strong | % Strong | | strong | % Strong |
| | adhesion | adhesion | | adhesion | adhesion |
| Sample | (>1.5 nN) | (>1.5 nN) | Sample | (>1.5 nN) | (>1.5 nN) |
| No | 0/239 | 0 | No | 0/239 | 0 |
| Bacteria | | | Bacteria | | |
| Polished | 23/257 | 8.9 | Polished | 26/191 | 13.6 |
| control | | | control | | |
| Run 24 | 20/227 | 8.8 | Run 24 | 10/187 | 5.3 |
| (18) | | | (18) | | |
| Run 25 | 36/187 | 19.3 | Run 25 | 29/241 | 12.0 |
| (21) | | | (21) | | |

The results indicate that Run 18 encountered the least number of strong adhesions from AFM probing of either strain of bacteria.

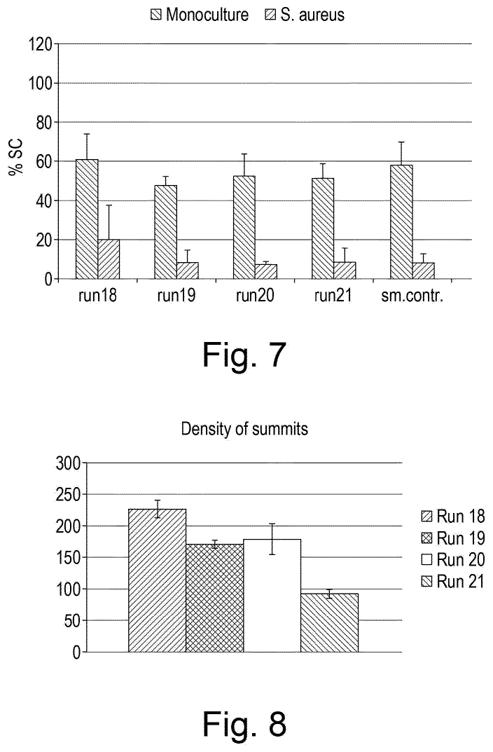

A ‘race to the surface’ test was conducted, used to determine the rate at which an introduced bacterial strain colonizes and envelopes a surface in competition with human mesenchymal stem cells (hMSCs). This is measured by quantifying the surface coverage of hMSCs following 24 hours of dual incubation of bacteria and hMSCs. In this test Ti6Al4V surfaces were exposed to S. aureus bacteria suspensions (5×10² bacteria/ml) for 60 minutes under 100% humidity. Samples were removed from the suspensions, removing any un-attached bacteria. hMSCs were seeded on the bacterial-coated samples (3×10⁴ cells/ml) and maintained at 37° in a humidified 5% CO₂ atmosphere for 24hrs. The hMSC surface coverage at the 24 hour time point is presented in FIG. 7 along with a control sample (sm.contr.).

From analysis of the results it appears that Run 18 is most successful at resisting the proliferation of the S. aureus bacteria strain at the 24hr period. It is important to keep in mind that in these tests, if given sufficient time, the bacterial strain is inevitably going to out-compete the hSMCs for coverage of the surfaces. This is due to the virility of the bacterial strain and the lack of immune response available to resist surface biofilm formation.

FIG. 8 shows the density of peaks (per μm²) for the four surfaces shown in FIGS. 3 to 6. All the surfaces have a peak density in a range 50 to 500 peaks per μm² with the most successful sample, sample 18, having a density of peaks of approximately 225 peaks per μm² representing a preferred value for this parameter, at least within the context of this study.

The data of FIG. 8 was generated using analysis software which derives summits from peaks in the surface height data. The analysis software defines a peak as any point, above all eight nearest neighbours. Summits are constrained to be separated by at least 1% of the minimum “X” or “Y” dimension comprising the 3D measurement area. Additionally, summits are only found above a threshold that is 5% of the depth at the lowest point above the mean plane.

FIG. 9 shows the average feature size (nm) for the four surfaces shown in FIGS. 3 to 6. All surfaces have an average feature size in a range 25 nm to 65 nm with the most successful sample, sample 18, having an average feature size of approximately 35 nm representing a preferred value for this parameter, at least within the context of this study. In this regard, the average feature (or grain) size is calculated using commercially available software (Gwyddion software) following an image processing threshold operation which segments the image in order to identify representative grains, exclude very small features, and then calculate the average feature size by averaging the equivalent square size of the identified grains.

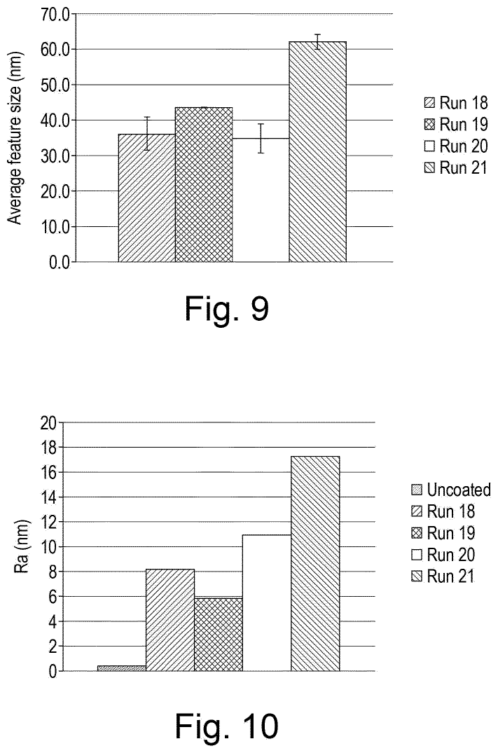

FIG. 10 shows the surface roughness Ra (nm) for the four surfaces shown in FIGS. 3 to 6. All surfaces have a surface roughness (Ra) in a range >5 nm to 18 nm with the most successful sample, sample 18, having a surface roughness of approximately 8 nm representing a preferred value for this parameter, at least within the context of this study.

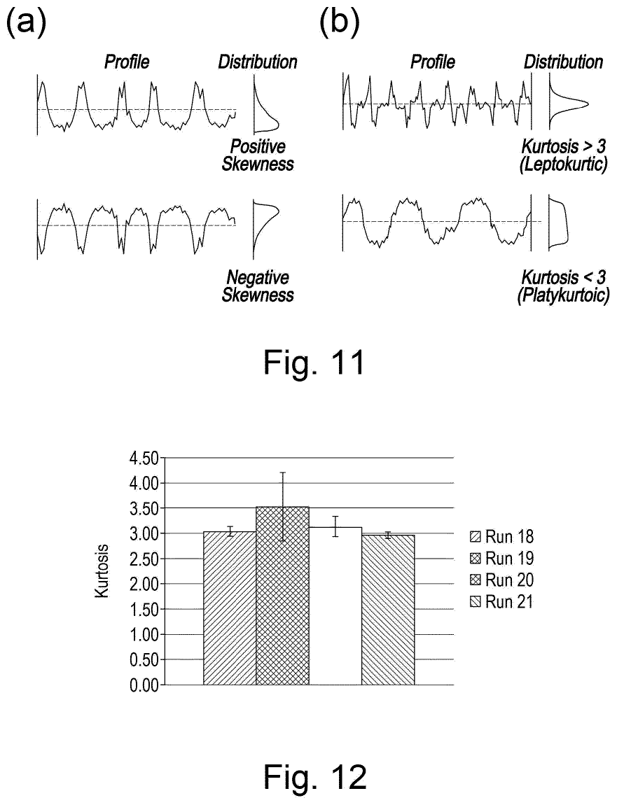

FIGS. 11(a) and 11(b) illustrated the surface parameters of skewness and kurtosis respectively. As previously described in the summary section, skewness is a measure of asymmetry in a histogram of projection height distribution whereas kurtosis is a measure of whether the surface is peaked or flat relative to the mean.

FIG. 12 shows kurtosis data for the four surfaces shown in FIGS. 3 to 6. All surfaces have a kurtosis in a range 2.50 to 4.00 with the most successful sample having a value of approximately 3.00 which represents a preferred value for this parameter, at least within the context of this study.

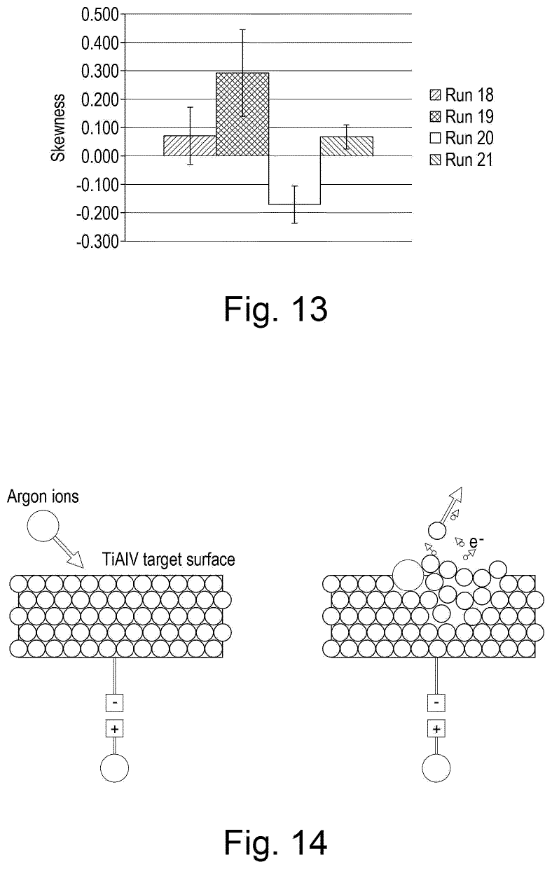

FIG. 13 shows skewness data for the four surfaces shown in FIGS. 3 to 6. All surfaces have a skewness within a range −0.20 to +0.30 with the most successful sample, sample 18, having a value of approximately 0.075 which represents a preferred value for this parameter, at least within the context of this study.

In addition to the above, it should also be noted that sample 18 in particular exhibits rounded peaks. As previously indicated, the combination of a relatively high peak density in combination with rounded peaks, and the fact that staphylococcus bacterial bodies are generally spherical and relatively rigid, actually results in low bacterial adhesion. Furthermore, the surface is more amenable to adhesion of larger, deformable host cells which can result in an overall improvement in performance.

As described in the summary section, surfaces as described herein can be formed by a coating on an implant body. However, it is also envisaged that such surfaces can be formed directly into the implant body by, for example, etching. The surfaces can be formed of titanium or a titanium alloy such as a titanium aluminium vanadium alloy. Such materials are consistent with those used presently for implant bodies such as prosthetic joints and components thereof.

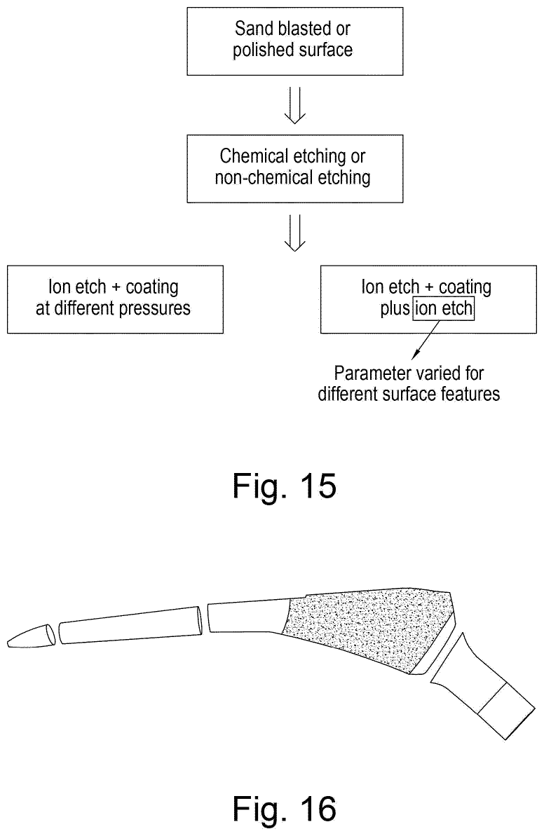

One such method utilizing a sputtering coating technique. FIG. 14 illustrates a sputtering coating technique. An ion source directs argon ions into a target which can be formed of titanium or a titanium alloy and which functions as a sputtering source. Atoms are ejected from the target and coated on a suitably placed substrate, which in the present case is an implant body such as prosthetic joint or a component thereof. The method can utilize etching (e.g. chemical and/or ion etching), passivation, and coating steps. FIG. 15 illustrates the surface processing steps including etching and coating. The table below lists a number of different processes used to fabricate samples 18 to 21 which have been previously discussed.

| | | | Ion etch | |
|---|---|---|---|---|
| | Chemical | | during | |
| process | etching | passivation | deposition | Run number |
| 1 | x | x | ✓ | 18, 24 |
| 2 | x | x | x | 19, 22 |
| 3 | ✓ | x | x | 20, 23 |
| 4 | ✓ | ✓ | x | 21, 25 |

The specific conditions for each step can be tailored to achieve the desired final surface finish. The specific operational parameters values will vary according to the equipment used. However, a person skilled in the art will be able to tune the operating parameters to achieve a desired final surface finish relatively easily given the teachings as provided herein and their common general knowledge of etching and deposition equipment. The critical feature is knowing what surface structure is desired for a particular application.

FIG. 16 shows a stem of a prosthetic hip joint to which the surface processing has been applied. The part has been segmented to allow for better characterisation of the surface structure. The stem comprises both non-porous regions and also regions formed of a porous coating to promote bone adhesion and ingrowth. Coatings as described herein can be applied to both the porous and non-porous regions of the medical implant. Furthermore, it is possible to reliably coat relatively complex three dimensional implant components compared to prior art methods of fabricating nano-columnar surfaces using a glancing angle deposition technique.

The present specification thus enables the provision of advanced titanium implants with controlled nanotopographies for dual regulation of bacterial and mammalian cell adhesion. While the invention has been described in relation to certain embodiments it will be appreciated that various alternative embodiments can be provided without departing from the scope of the invention which is defined by the appending claims.

1. A medical implant comprising:
an implant both configured for use as a medical implant,
a surface on the implant body,
wherein the surface comprises a plurality of projections, each projection having a base proximal to the implant body, a peak distal to the implant body, and a side wall extending from the base to the peak,
wherein the surface has a peak density in a range 50 to 500 peaks per μm2, and
wherein the projections are tapered such that a width at the peak of each projection is less than a width at the base of each projection.

2. A medical implant according to claim 1,
wherein the peak density is greater than 100, 150, or 200 peaks per μm2.

3. A medical implant according to claim 1,
wherein the peak density is less than 400, 300, or 250 peaks per μm2.

4. A medical implant according to claim 1,
wherein the surface has a kurtosis in a range 2.50 to 4.00.

5. (canceled)

6. (canceled)

7. A medical implant according to claim 1,
wherein the surface has a skewness in a range −0.20 to +0.30.

8. (canceled)

9. (canceled)

10. A medical implant according to claim 1,
wherein the surface has a surface roughness (Ra) in a range >5 nm to 18 nm.

11. A medical implant according to claim 10,
wherein the surface roughness (Ra) of the surface is greater than 6 nm, 7 nm, or 7.5 nm.

12. A medical implant according to claim 10,
wherein the surface roughness (Ra) of the surface is less than 14 nm, 12 nm, 10 nm, or 9 nm.

13. A medical implant according to claim 1,
wherein the surface has an average feature size in a range 25 nm to 65 nm.

14. A medical implant according to claim 13, wherein the average feature size of the surface is greater than 30 nm.

15. A medical implant according to claim 13,
wherein the average feature size of the surface is less than 55 nm, 45 nm, or 40 nm.

16. A medical implant according to claim 1,
wherein the width at the base of each projection is at least 1.2, 1.4, 1.6, 1.8 or 2 times the width of each projection at ⅘th of a height of each projection.

17. A medical implant according to claim 1,
wherein the peak of each projection is rounded.

18. A medical implant according to claim 17,
wherein the rounded peak of each projection has a radius of curvature in a range 5 nm to 200 nm.

19. A medical implant at to claim 1,
wherein the projections having a height from base to peak in a range 30 nm to 90 nm.

20. (canceled)

21. (canceled)

22. A medical implant according to claim 1,
wherein the surface is formed by a coating on the implant body.

23. A medical implant according to claim 1,
wherein the surface is formed of titanium or a titanium alloy.

24. (canceled)

25. A medical implant according to claim 1,
wherein the implant body is a prosthetic joint or a component thereof.
